(19) United States
(12) Patent Application Publication
(10) Pub. No.: US 2008/0268429 A1
(43) Pub. Date: Oct. 30, 2008

(54) Rna - Containing Microvesicles and Methods Therefor

(75) Inventor: Zbigniew Pietrzkowski, Dyer, IN (US)

Correspondence Address: FISH & ASSOCIATES, PC;ROBERT D. FISH, 2603 Main Street, Suite 1050, Irvine, CA, 92614-6232, US

(73) Assignee: SOURCEPHARM, INC., San Diego, CA (US)

(21) Appl. No.: 11/569,757

(22) PCT Filed: Mar. 30, 2005
(86) PCT No.: PCT/US2005/010674

§ 371 Date: Jan. 10, 2008

Related U.S. Application Data
(60) Provisional application No. 60/576,395, filed on Jun. 2, 2004.

(51) Int. Cl. C12Q 1/68

(52) U.S. Cl. 435/6

(57) ABSTRACT

Contemplated compositions and methods are directed to the use of microvesicles from an optionally recombinant donor cell to impart a desirable effect to a recipient cell. In certain preferred aspects, RNA of the microvesicles is employed to achieve the desirable effect. For example, microvesicles are used in vitro to increase the number of passages of a cell growing in a medium, reduce serum and/or growth factor requirements of a cell growing in a medium, and/or delay differentiation of a cell growing in a medium. Further preferred aspects include use of the microvesicles as therapeutic agents in which RNA, a membrane protein, and/or a cytosolic protein encapsulated in or coupled to the microvesicle provide a therapeutic effect. Additionally, diagnostic methods are contemplated in which RNA of a microvesicle isolated from a mammal is associated with a condition of the mammal.

This application claims the benefit of our U.S. Provisional Patent Application with the Ser. No. 60/576,395 (filed Jun. 2, 2004), which is incorporated by reference herein.

### FIELD OF THE INVENTION

The field of the invention is biotechnology, and especially as it relates to compositions and methods for microvesicles.

### BACKGROUND OF THE INVENTION

Microvesicles were historically regarded as cellular debris with no apparent function. However, and more recently, a growing body of experimental data suggest that microvesicles have numerous biological activities. For example, platelet-derived microvesicles were shown to stimulate selected cells via surface proteins on the microvesicles (e.g., CD154, RANTES, and/or PF-4; see Thromb. Haemost. (1999), 82:794, or J. Biol. Chem. (1999), 274:7545). In other examples, specific effects of bioactive lipids (e.g., sphingosine-1-phosphate, HETE, or arachidonic acid) in platelet microvesicles on certain target cells were reported (see e.g., J. Biol. Chem. (2001), 276: 19672; or Cardiovasc. Res. (2001), 49(5):88). In further examples, platelet microvesicles increased adhesion of mobilized CD34+ endothelial cells by transfer of certain microvesicle surface components to the mobilized cells (see Blood (2001), 89:3143).

Based on these and other discoveries, various clinical uses were proposed for platelet-derived microvesicles, including wound healing as disclosed in U.S. Pat. No. 5,428,008, hemostasis as taught in U.S. Pat. No. 5,165,938, and tissue regeneration as described in U.S. Pat. App. No. 2004/0082511. While such uses provide at least some promising perspectives, several largely unexplained problems remain. Among other things, biological activity is often difficult to predict. Moreover, therapeutic use generally necessitates sterilization and antiviral treatment to prevent infections of the people receiving microvesicle containing preparations.

Thus, while numerous compositions and methods for microvesicles are known in the art, all or almost all of them, suffer from one or more disadvantages. Most significantly, and among other things, predictability of biological effects in many uses of such microvesicles is problematic. Therefore, there is still a need for improved compositions and methods as they relate to microvesicles from nucleated and non-nucleated cells.

### SUMMARY OF THE INVENTION

The present invention is directed to compositions and methods in which microvesicles from a donor cell (or synthetic microvesicles) are employed to modify a cell that is contacted with the microvesicles. Most typically, the modification is at least in part effected by RNA of the microvesicle, but may also be (alternatively or additionally) effected by a lipid component of the microvesicle, a membrane associated peptide of the microvesicle, or a cytosolic peptide of the microvesicle. Depending on the donor cell and/or the timing of preparation of the microvesicle, it should be recognized that the contents and composition of the microvesicle may vary. Therefore, effects on contemplated recipient cells may vary considerably.

In one aspect of the inventive subject matter, a kit includes a composition comprising a microvesicle from a donor cell, wherein the microvesicle encloses at least one RNA, and further includes an information associated with the composition that provides at least one of a function and an identity of the RNA. Thus, methods of modifying a cell are contemplated in which a plurality of microvesicles are provided that enclose RNA known to confer a specific effect. In another step of such methods, a recipient cell is contacted with the microvesicles in an amount sufficient to deliver the RNA into the recipient cell to thereby modify the recipient cell.

In especially preferred kits and methods, the donor cell is a stem cell, a differentiated cell, a diseased cell, and/or an apoptotic cell, which may optionally be treated or recombinant. Recombinant donor cells may include a recombinant nucleic acid that encodes a regulatory RNA, a secreted or a membrane associated protein. Depending on the nature of the donor cell, contemplated microvesicles may further have a membrane associated protein, a cytoplasmic protein, a nuclear protein, and a membrane lipid component, wherein these components preferably exert a desirable effect on a recipient cell that is contacted with the microvesicle. Consequently, a method of marketing a product has a step in which a composition is provided that comprises a microvesicle from a donor cell, wherein the microvesicle has at least one RNA. In another step of such methods, an information is provided and associated with the composition that provides at least one of a function and an identity of the RNA.

In another aspect of the inventive subject matter, a kit includes a composition that comprises a (preferably preserved) microvesicle from a donor cell. Such kits may further include an information associated with the composition to combine the microvesicle with a recipient cell in vitro to achieve a desired effect in the recipient cell. Among other desired effects, especially preferred effects include an increased number of passages, an increased susceptibility to viral infection, a delay in differentiation, a reduction in requirements for serum, and/or a reduction in requirements for a growth factor. Such desired effects may be effected by, among other things, RNA, a membrane associated protein, a cytoplasmic protein, a nuclear protein, and/or a membrane lipid component of the microvesicle.

In a still further aspect of the inventive subject matter, it is contemplated that a kit may also include a composition that includes a preserved (e.g., dehydrated) microvesicle from a donor cell, and an information associated with the composition to combine the microvesicle with an in vitro growth medium to achieve a desired effect. Among other desired effects, an increased number of passages of a cell growing in the medium, reduction in requirements for serum and/or growth factors of a cell growing in the medium, and/or a delay in differentiation of a cell growing in the medium are especially contemplated. Thus, particularly contemplated aspects also include powdered media formulations that include a preserved microvesicle. Depending on the use, it is generally contemplated that a kit will include a composition that comprises a preserved microvesicle from a donor cell, and an information associated with the composition to use the composition for an in vitro use.

In yet another aspect of the inventive subject matter, the inventors contemplate a method of diagnosing a condition in a mammal in which in one step a sample is provided that includes a plurality of microvesicles isolated from the mammal, wherein at least one of the microvesicles encloses an RNA. In another step, presence and/or quantity of the RNA in the sample is determined, and in yet another (optional) step, the presence and/or quantity of the RNA is correlated with the condition. Typically, the sample will include a biological fluid of the mammal, and the step of determining presence/quantity of the RA will include RT-PCR, Q-PCR, and/or hybridization of the RNA on a solid phase.

While not limiting to the inventive subject matter, it is contemplated that the RNA in such methods has a sequence associated with at least one of a viral infection (e.g., HIV, HCV, or poxviridae infection), a metabolic disease (e.g., PKU, hypoketotic hypoglycemia, glycogen storage disease), and of a neoplastic disease (e.g., prostate, colon, or breast cancer).

Various objects, features, aspects and advantages of the present invention will become more apparent from the following detailed description of preferred embodiments of the invention.

### BRIEF DESCRIPTION OF THE DRAWINGS

FIG. 1 is a graph depicting relative quantities of selected RNA species in donor cells and microvesicles from the donor cells.

FIG. 2 is an autoradiograph demonstrating RNA integrity from lyophilized microvesicles.

FIG. 3 is an autoradiograph demonstrating gene expression in a recipient cell from RNA provided by microvesicles and RNA integrity after RNAse treatment of microvesicles.

FIG. 4 is a graph depicting difference in expression of various genes in cells treated with ES microvesicles versus control cells treated with growth factors.

FIG. 5 is a graph illustrating an increase in clonogenicity of cells treated with ES microvesicles versus control cells treated with growth factors.

### DETAILED DESCRIPTION

The inventors have unexpectedly discovered that microvesicles from nucleated cells and/or from non-nucleated cells can be employed to modify recipient cells in a predetermined manner. Most typically, such modification is at least in part mediated by nucleic acids, and particularly RNA contained in the microvesicles, which was heretofore not recognized. Exemplary Scheme 1 below depicts the general concept contemplated herein:

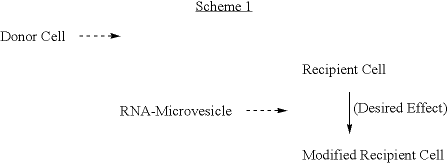

The inventors still further discovered that RNA-containing microvesicles may also be employed in numerous diagnostic and therapeutic applications. In such instances, it should be recognized that the specific RNA content of the microvesicle will determine the diagnosis and/or the therapeutic use.

The term “microvesicle” as used herein refers to a membranaceus particle having a diameter (or largest dimension where the particle is not spheroid) of between about 10 nm to about 5000 nm, more typically between 30 nm and 1000 nm, and most typically between about 50 nm and 750 nm, wherein at least part of the membrane of the microvesicle is directly obtained from a cell. Therefore, especially contemplated microvesicles include those that are shed from a donor cell, and will typically include exosomes and synaptosomes. In contrast, a liposome that is fabricated from characterized lipids and does not include lipids obtained from a cell-membrane is not considered a microvesicle under the definition used herein.

As also used herein, the term “donor cell” refers to any cell from which a microvesicle is obtained. As further used herein, the term “recipient cell” refers to any cell that at least partially fuses with a microvesicle. As used herein, the term “at least partially fuses” refers to a process in which at least part of the lipid layer of the microparticle becomes temporarily or permanently a portion of at least part of the lipid cell membrane of the recipient cell. Therefore, the term “at least partially fuses” includes attachment of the microvesicle to the outer cell membrane, fusion of the microvesicle with the outer cell membrane (with or without concurrent release of the microvesicle content to the inside of the recipient cell), ad endocytosis.

Contemplated Donor Cells

It should be recognized that the nature of the donor cell is generally not limiting to the inventive subject matter so long as a microvesicle can be obtained from the donor cell. Thus, suitable cells include prokaryotic cells, archaebacterial cells, fungal cells, and single- and multi-cellular eukaryotic cells.

Where needed, it should be appreciated that bacterial and/or yeast cell walls can be removed before obtaining microvesicles from such organisms. Alternatively, microvesicles can be obtained from bacteria known to shed microvesicles, including Agrobacterium spp., Bacillus spp., Borrelia spp., Bordetella spp., Calymmatobacterium spp., Escherichia spp., Haemophilus spp., Neisseria spp., Pseudomonas spp., Porphyromonas spp., Fusobacterium spp., Rhodococcus spp., Salmonella spp., Serratia spp., Shigella spp., and Yersinia spp. In most cases where the microvesicles are obtained from a bacterium, the bacterial cell may be transfected to provide at least one of a recombinant membrane protein and a recombinant RNA in the microvesicle. Furthermore, where the lipid composition of the bacterial and/or fungal cell reduces or even inhibits incorporation, fusion, and/or attachment of the microvesicle to the recipient cell, it is contemplated that the bacterial or fungal cell may be modified with a compound that increases incorporation, fusion, and/or attachment of the microvesicle to the recipient cell. For example, suitable compounds may be cationic detergents, polymeric compounds (e.g., PEG), and/or polypeptides on the surface of the microvesicle.

Where the donor cell is a eukaryotic cell, it is generally preferred that the eukaryotic is a multi-cellular organism, and more preferably a vertebrate cell (e.g., mammal). Typically, suitable donor cells will produce the microvesicles spontaneously, or in response to chemical and/or mechanical stimuli. For example, various embryonic and other stem cells are known to spontaneously produce significant quantities of microvesicles. On the other hand, cytokine and/or mechanical stimulation of thrombocytes results in substantial increase in microvesicle formation. Furthermore, it should be recognized that the donor cell may be nucleated or non-nucleated. Thus, suitable donor cells include lymphocytes (e.g., polynucleated, polymorpho-nuclear lymphocytes, etc), fibroblasts, hepatocytes, as well as erythrocytes, and thrombocytes. Additionally contemplated cells may also have lost at least part of the genomic information relative to a totipotent stem cell from which they were derived (e.g., aneuploid neural or glial cell), which may be a consequence of natural development or disease.

Similarly, it should be recognized that suitable donor cells may be derived from any desirable developmental stage with respect to its cell lineage. Consequently, suitable donor cells especially include stem cells (which may or may not be committed to a particular cell line), partially differentiated stem cell, and fully differentiated cells. Moreover, donor cells contemplated herein may be in any stage of their individual cellular age, ranging from just separated from their progenitor cell to a senescent or even dead cell. Remarkably, increased shedding of microvesicles is at times associated with apoptotic blebbing (which may be from the plasma membrane and/or the nucleus). Thus, pre-apoptotic donor cells, or cell committed to apoptosis are also contemplated herein.

Furthermore, it is contemplated that suitable donor cells also include non-diseased and diseased cells, wherein diseased cells may be affected by one or more pathogens and/or conditions. For example, a diseased donor cell may be infected with a virus, an intracellular parasite, or bacterium. In other examples, the diseased cell may be a metabolically diseased cell (e.g., due to genetic defect, due to an enzyme, receptor, and/or transporter dysfunction, or due to metabolic insult), a neoplastic cell, or cell that has one or more mutations that render the cell susceptible to uncontrolled cell growth. Similarly, donor cells may be native (e.g., obtained by biopsy), cultured (e.g., native, or immortalized), or treated. For example, donor cells may be chemically and/or mechanically treated, resulting in a donor cell that exhibits a cell-specific stress response. In another example, contemplated donor cells may be treated with a natural or synthetic ligand to which the cell has a receptor or otherwise complementary structure. In still further examples, the donor cell may also be treated with a drug or compound that alters at least one of a metabolism, cell growth, cell division, cell structure, and/or secretion.

Additionally contemplated treatments include those in which one or more nucleic acid molecules are introduced into the cell to thereby form a recombinant cell. It should be noted that all known manners of introducing nucleic acids are deemed suitable for use herein (e.g., viral transfection, chemical transfection, electroporation, ballistic transfection, etc.). Where the nucleic is a DNA, it is contemplated that the DNA may be integrated into the genome of the donor cell, or that the DNA may reside as extrachromosomal unit within the cell. Such DNA may be employed as a template for RNA production, which may have regulatory and/or protein encoding function. Similarly where the nucleic acid is an RNA, such RNA may be used as a regulatory entity (e.g., via antisense or interference) and/or as a protein encoding entity. Of course, all known nucleic acid analogs (e.g., phosphorothioate analogs, peptide nucleic acid analogs, etc.) are also deemed suitable for use herein.

With respect to the origin of the donor cell, it is contemplated that the donor cell may have any origin that will be desired, including endothelial, mesothelial, and ectothelial origin. Thus, suitable donor cells will include those commonly found in a gland, an organ, muscle, a structural tissue, etc. Furthermore, it should be recognized that contemplated donor cells may be heterologous or autologous relative to recipient cell. For example, a donor cell may be a porcine pancreatic cell, while the recipient cell is a human pancreatic cell. Similarly, a donor cell may be a fibroblast from a one human, while the recipient cell is a fibroblast of another human. Where the donor cell and the recipient cell are autologous, it should be particularly appreciated that the recipient cell may be aged relative to the donor cell. For example, a stem cell at an early culture stage may be employed as a donor cell for the same stem cell at a culture stage in which the donor cell has undergone at least one subsequent cell division.

Contemplated Microvesicles

Based on the above considerations on suitable donor cells, it should be recognized that the structure and composition of contemplated microvesicles will vary substantially, and that the type and condition of the donor cell as well as the preparation of the microvesicles will at least in part determined the characteristics of the microvesicles.

### Microvesicles Isolated from a Donor Cell

Most typically, microvesicles can be isolated by numerous manners well known in the art, and especially preferred methods include centrifugal isolation from biological fluids or cell culture supernatants. Exemplary methods for isolation of microvesicles are described in Platelets (2004), 15(2): 109-115, J. Immunol. (1998), 161(8):4382-4387, or in Curr. Opin. Hematol. (2004), 11(3): 156-164. Alternatively, microvesicles may also be isolated and/or characterized via flow cytometry as described in Thromb Haemost. (1997), 77(1): 220. Most commonly, contemplated microvesicles will have a size (average diameter) that is up to 5% of the size (average diameter) of the donor cell. Therefore, and depending on the particular donor cell, the microvesicles will have an average size of 10 nm to about 5000 nm, more typically between 30 nm and 1000 nm, and most typically between about 50 nm and 750 nm. In still further contemplated aspects, it should be appreciated that isolation of microvesicles need not be limited to cell culture supernatants, but that all biological fluids (including whole blood, urine, milk, saliva) and biopsy samples are also deemed suitable.

With respect to membrane composition of contemplated microvesicles, it is generally contemplated that the membrane composition reflects the average membrane composition of the donor cell. Therefore, the exact membrane composition will in large degree depend on the particular membrane composition of the donor cell. However, it should be recognized that due to the specific manner of microvesicle shedding, the membrane composition may be also be representative of only a local membrane compositions of the donor cell. For example, while contemplated microvesicles typically have the same structure (i.e., the lipid bilayer membrane, typically only in the form of a single vesicular structure and not in the form of nested vesicles) as the donor cell, various membrane components or molecules associated with the membrane may not or predominantly be found in the donor cell or microvesicle.

Among other components that may be unevenly distributed between the microvesicle and the donor cell are lipid-based molecules e.g., glycolipids, ceramide, etc.), transmembrane proteins, receptors, transporters, components functionally associated with signal transduction across the membrane, etc. For example, asymmetric glycolipids distribution was reported in Acta Biochemica Polonica (1998), 45(2), while chemokine receptor loading of microvesicles was reported in Nat. Med. (2000), 6(7):769-75. Similarly, intracellular peptides may be found in microvesicles, either evenly distributed, or unevenly between donor cell and microvesicle as reported for Il-1 beta in Proc Natl Acad Sci (2004), 101(28): 10241-10242.

Furthermore, it should be especially recognized that the microvesicles from the donor cells will also include nucleic acids that represent an average, and/or specific population of the nucleic acids found in the donor cell. Depending on the particular donor cell type, the nucleic acids will include DNA and/or RNA, wherein RNA expressly includes heterogenous nuclear RNA (hnRNA), optionally capped and/or polyadenylated messenger RNA (mRMA), transfer RNA (tRNA), small interfering RNA (siRNA), intron-corresponding RNA (icRNA), and ribosomal RNA (rRNA). Such RNA will typically be reflective of at least one of the cell type, cell age, cell condition (e.g., healthy or diseased), and cell source. Additionally, such RNA may also be derived from a recombinant DNA that was previously introduced into the donor cell. It should be especially noted that at least some of the RNA in the microvesicles may be preferentially or even be selectively present or absent in the microvesicles. Among other things, it is known that RNA and DNA is segregated into separate apoptotic bodies as discussed in Exp. Cell Res. (2000), 260: 248-256. Furthermore, RNA may also be selectively subjected to microvesicle export via RNA trafficking as reported in Biol. Cell (2005), 97: 5-18. Based on the addressability of RNA to localize with the cell membrane, it is especially contemplated that recombinant DNA in a donor cell may include a signal that prompts the RNA to be located with the cell membrane (see e.g., Dev. Cell (2003), 5:161-174).

Therefore, and at least from this perspective, it should be recognized that the RNA in microvesicles may provide a recipient cell (of the same or different tissue type) with one or more molecules that will interact with the recipient cell in a predetermined or characteristic manner. For example, where the RNA in the microvesicle is a mRNA, the recipient cell may be modified by the microvesicle to express the corresponding polypeptide even if the recipient cell would otherwise not produce that protein. In another example, where the RNA in the microvesicle is siRNA, the recipient cell may be modified by the microvesicle to up- or down-regulate expression of a responsive gene in the recipient cell. In a still further example, where the RNA in the microvesicle is rRNA, the microvesicle may down-regulate ribosomal synthesis and with that translation in the recipient cell.

Consequently, it should be recognized that the RNA of the microvesicle may be employed to achieve a desired effect in the recipient cell via at least one RNA (and most typically an RNA that is characterized). Viewed from another perspective, it should be recognized that the a microvesicle will include an RNA known to confer a specific effect to a recipient cell. The term “RNA known to confer a specific effect” as used herein refers to an RNA that has been demonstrated (in vivo, in vitro, or in silico) to affect a recipient cell in a predetermined manner, wherein the effect may be a change in a single parameter (e.g., increase or decrease in expression of a specific gene), a plurality of parameters (e.g., modification in a signal transduction, or change in developmental progress [e.g., delay in differentiation]), or affect the entire cell (e.g., induce apoptosis, differentiation, etc.).

Moreover, in further aspects of the inventive subject matter, it is contemplated that the RNA in the microvesicle will provide the desired effect on the recipient cell in combination with at least one other factor that is associated with the microvesicle, and especially preferred factors include membrane associated polypeptides (e.g., receptors, channels, polypeptides in signal transduction across a membrane), soluble polypeptides (e.g., cytokines, transcription factors, etc.), and lipid composition (e.g., glycosphingolipids, glycerophospholipids, etc). Therefore, it should be recognized that microvesicles can be employed as an antigen against a cell from which the microvesicle has been isolated and thereby generate an immunological response in a host challenged with the microvesicles. In such a case, the effect of the microvesicle is indirect or extended (e.g., the recipient cells are immunological cells and after priming with microvesicles these immunological cells affect donor cells directly or though antibodies).

### Synthetic Microvesicles

In alternative aspects of the inventive subject matter, it is also contemplated that the microvesicles may be synthetically produced, wherein the synthetic microvesicle preferably includes one or more membrane components obtained from a donor cell. However, in some alternative aspects, the membrane composition may be simulated to resemble at least in some respect the membrane composition of a particular donor cell. Regardless of the manner of preparation, it is typically preferred that the synthetic microvesicle includes at least one RNA. For example, synthetic microvesicles may be prepared by disintegration of a donor cell (e.g., via detergent, sonication, shear forces, etc.) and use of the crude preparation or an at least partially enriched membrane fraction to reconstitute one or more microvesicles.

Using a synthetic route of microvesicle preparation, it is contemplated that the RNA in such microvesicles may be genuine to the donor cell, or may be of recombinant origin (as part of a modified donor cell or exogenously added RNA). With respect to the RNA and the donor cell from which the membranes are obtained, the same considerations as provided above apply. Moreover, where the microvesicles are synthetic, it is contemplated that such microvesicles may be loaded with a pharmaceutically active molecule (e.g., in a similar fashion as one would load liposomes).

### Microvesicle Preparations

Microvesicles contemplated herein are preferably isolated from their donor cells in, or synthesized and isolated from a physiologically acceptable solution. For example, suitable solutions include buffered saline, growth medium, various aqueous media, etc. Typically, the microparticles will be present in such solutions at a concentration of about 10² to about 10⁸ particles per milliliter, or even more. Depending on the particular use, the microparticles may then be characterized in their RNA content (e.g., via solid phase hybridization, qPCR, etc.), and further characteristics (e.g., content of membrane associated polypeptides, composition of lipids, content/composition of soluble polypeptides, etc.) may be determined as appropriate.

Additionally, the microvesicles contemplated herein may be modified to achieve a particularly desirable result. For example, suitable modifications include those in which one or more membrane associated components are at least partially removed to reduce potential immunogenicity of the microvesicles where the microvesicle is introduced (directly or indirectly via recipient cells) into a mammal. For example, removal may be performed using various enzymes (e.g., proteases, glycosylases), or chemically. Similarly, it is contemplated that membrane associated components may be added to the microvesicle (e.g., via fusion with another microvesicle) to induce immunotolerance, or to assist in homing in of microvesicles or cells treated with microvesicles to a target tissue. Moreover, one or more independent preparations of microvesicles may be fused (e.g., using ultrasound and/or PEG) to combine two or more desirable properties, which may or may not be associated with the mRNA content. For example, microvesicles from two distinct preparations may be fused to achieve a particular lipid and/or protein profile. Alternatively, microvesicles may also be fragmented. Regardless of the nature and/or origin of the microvesicles, it is generally contemplated that the microvesicles may be labeled for use in basic research or diagnostics, or to allow monitoring fate and/or location of the microvesicles. Suitable labeling agents include all known labeling agents known in the art, and especially preferred labeling agents include GFP, Luciferase, microbeads, antibodies, and radiolabels.

In further particularly preferred aspects on the inventive subject matter, microparticles (e.g., isolated from donor cells or synthetic) are treated and/or prepared in a preparation that increases physical and/or chemical stability. For example, where the microparticles remain in an aqueous (or other) medium, it is generally preferred to quick-freeze the preparation to a temperature of between about −60° C. to about −80° C. (or even lower temperature). In these cases, the microparticle solution may include one or more preservatives (e.g., NaN₃, benzoate, etc.), antioxidants, and/or cryoprotectants that reduce damage to the freezing preparation (e.g., glycerol, DMS, propanediol, etc.). Alternatively, contemplated microvesicles may also be dehydrated (e.g., freeze-dried, spray dried, etc.), wherein the dehydrated microvesicles may include lyoprotectants (e.g., trehalose, serum protein, sucrose, hydroxymethyl starch, etc.), antioxidants, etc.

Where desirable, isolated and/or dehydrated microparticles may also be incorporated into numerous pharmaceutical and/or cosmetic preparations, and the particular use will eventually determine the specific formulation. For example, where the microparticles are employed as a therapeutic agent (e.g., for vaccination), suitable formulations may be in form of an injectable or sprayable formulation, or in topical formulation (e.g., in form of eye drops). In another example, where the microvesicles are employed as a wound healing agent, suitable formulations may include those in which the microvesicles are part of a wound dressing (e.g., in a band aid or gauze). In less preferred aspects, the microvesicles may also be formulated for oral administration. Alternatively, where the microvesicles are employed for cosmetic purposes, preferred formulations include topical formulations, most preferably in form of a solution, cream, lotion, ointment, or lipstick. There are numerous compositions and methods for pharmaceutical and cosmetic formulations known in the art, and all of those are considered suitable for use herein.

Contemplated Recipient Cells

It is generally contemplated that all cells and cell-containing structures, and especially living cells and structures comprising same are suitable as recipient cells and/or recipients for the microvesicles according to the inventive subject matter.

Therefore, with respect to the cell type (e.g., prokaryotic cells, archaebacterial cells, fungal cells, and single- and multi-cellular eukaryotic cells), cell origin (e.g., endothelial, mesothelial, ectothelial), cell age (e.g., just separated from parent cell, between 2-8 divisions or older, committed to apoptosis, etc), stage of differentiation (e.g., undifferentiated stem cell, stem cell committed to lineage, partially differentiated, or fully differentiated), genomic content (e.g., euploidic, aneuploidic), and health condition (e.g., infected with a virus or bacterium, neoplastic, etc.), the same considerations as provided above. Furthermore, contemplated recipient cells may also be recombinant cells which harbor extrachromosomal or genomic recombinant DNA.

In one preferred aspect of recipient cells, it is contemplated that the recipient cell is a cell in vitro, and that the cell is not further modified to increase contact, fusion, and/or uptake of the microvesicle with the recipient cell. However, it should also be appreciated that the in vitro cells may be treated to increase contact, fusion, and/or uptake of the microvesicle. For example, the recipient cell may be chemically treated with cationic or non-ionic detergents, or polymers (e.g., PEG). Alternatively, the cells may also be physically treated using heat shock or ultrasound to promote cell fusion.

In another preferred aspect of recipient cells, it is contemplated that the recipient cell is a cell in vivo, and that the cell is contacted with the microvesicles via a application fluid or biological fluid in the organism to which the microvesicles are administered. For example, in vivo/in situ administration may include topical or local administration, and suitable methods include spraying of a microvesicle formulation, ocular application, or instillation. Alternatively, systemic administration is also contemplated and especially include injection and oral administration. Regardless of the location of the recipient cell (i.e., in vitro or in vivo), it should be recognized that the recipient cell may be an isolated cell, a cell in culture with the same or other type of cells, or even in a tissue, organ, or whole organism.

Consequently, depending on the particular application and situation of the recipient cell, it should be contemplated that the recipient cell will be contacted with the microvesicles in a temporary, extended, or even permanent manner. For example, where the recipient cell is a cell in vitro, the medium may be enriched with the microvesicles for a period of between several minutes to several hours (or days, where the microvesicle is removed during media change or passage), while recipient cells in vivo will typically be exposed to the microvesicles for a duration that is determined at least in part by the serum half-life time of the vesicle (e.g., between several hours and several days). With respect to the amount of microvesicles relative to recipient cells it is generally contemplated that the particular desired effect will at least partially determine the ratio. However, in most cases the ratio of microvesicles to recipient cells will be between about 10⁷:1 to about 10⁻¹:1, more typically between about 10⁶:1 to about 10²:1, and most typically between about 10⁵:1 to about 10³:1. Of course, it should be noted that a recipient cell may be contacted with more than one type of microvesicle, and the particular desired effects will determined the number and types of microvesicles with which the recipient cell is being contacted.

While not limiting to the inventive subject matter, it is generally preferred that the recipient cell will fuse with the microvesicle such that at least some of the RNA in the microvesicle will be delivered to the cytoplasm, and that at least some of the membrane components in the microvesicle are integrated into the recipient cell membrane (plasma membrane). Alternatively, or additionally, it is also contemplated that some of all of the microvesicles may be taken up into the cell without fusion of the microvesicle to the plasma membrane. In such instances, it is contemplated that the microvesicle has a membrane composition (e.g., via lipid profile and/or membrane associated polypeptides) effective to allow fusion or uptake of the microvesicle to non-plasma membrane compartments of the cell, and especially to the nucleus, the mitochondrion, and the endoplasmatic reticulum. Typically, such microvesicle is then taken up via endocytosis or phagocytosis.

Contemplated Uses

It should be recognize that the particular use of contemplated microvesicles depends at least in part on the specific RNA in the microvesicle, wherein the RNA confers a desired effect to the recipient cell. Moreover, additional components of the microvesicle that confer a desired function include lipid composition of the membrane of the microvesicle as well as membrane associated polypeptides. Therefore, numerous uses are contemplated in which the RNA that is associated with the microvesicles has a desired effect on a recipient cell, and/or in which microvesicles are used as immunomodulatory entities. Alternatively, microvesicles may also employed in a diagnostic test in which the RNA in the microvesicles serves as a diagnostic marker. Microvesicles can be measured by their amount of RNA and/or protein content. In most cases, it is contemplated that the amount of microvesicles needed for a desired effect will be in a range 0.01 μg/mL to 1000 μg/mL (protein/mL) or 0.1 ng to 1 μg/ml of RNA.

### Modulatory and/or Therapeutic Uses

In vitro uses: In one aspect of the inventive subject matter, RNA in microvesicles is employed to confer a specific effect to a recipient cell in vitro. For example, it is especially contemplated that a recipient cell can be transiently transfected with an RNA-containing microparticle such that the recipient cell expresses a surface receptor that allows the recipient cell to be virally transfected. Such exemplary use is particularly advantageous where it is desired to introduce recombinant DNA into stem cells (and most preferably embryonic stem cells). Normally, viral gene transfer is very inefficient, if not impossible with numerous stem cells. However, where a microvesicle includes an RNA that encodes for a receptor that is used by the virus for entry into the cell, viral transfection is relatively simple and effective. Most advantageously, such genetic transformation is transient, and will not modify the stem cell's genome prior to viral transfection.

Similarly, and further expanding on exemplary uses with stem cells, it should be appreciated that transient expression of numerous cell surface receptors will retain various physiological characteristics genuine only to early stage and/or undifferentiated stem cells, wherein such cell surface receptors are involved in maintenance of the undifferentiated nature of the stem cell. Thus, microvesicles isolated fro early stage cultures of stem cells may be employed to maintain the undifferentiated nature of a stem cell in a later stage stem cell. Thus, among numerous other uses, RNA in the microvesicles may be used to increase number of passages of a cell (rejuvenation by use of microvesicles from young cells), to increase the susceptibility to a viral infection (e.g., via transient expression of a viral docking/entry protein), to delay differentiation, etc.

Additionally, temporal knock-out and/or knock-down nucleic acids may be introduced into a cell, where the genome otherwise would have to be permanently mutated. For example, where the RNA in the microvesicle has a regulatory effect on the recipient cell (e.g., RNA is antisense RNA or siRNA), temporary gene silencing may be performed without alteration of the recipient cell's genome.

Based on previous experimental data (see below), the inventors also surprisingly discovered that microvesicles from early culture cells, and especially microvesicles from stem cells can be added to a growth medium of another cell (stem cell or other partially or fully differentiated cell) to effect a reduction in the cell's requirements for serum, and/or a reduction in the cell's requirement for one or more growth factors. Thus, it should be recognized that the microvesicles may also be added to a cell-free or cell containing growth medium to achieve numerous desirable effects. Among other benefits, partial or entire replacement of serum and/or growth factors in a medium will substantially reduce cost of cell culture, and reduce likelihood of contamination of the medium with a virus, mycoplasm, or other pathogenic agent.

Consequently, and at this point only considering RNA effects in microvesicles, it should be recognized that microvesicles can be employed to temporarily modify a cellular parameter, and especially expression of one or more genes (via down-regulation and/or introduction of a mRNA), presentation of a membrane associated polypeptide, which in turn may affect the cell's susceptibility to an external and/or internal signal, or infection with a virus, or modification of the cell's immunological profile (e.g., the cell may turn “invisible” or become recognizable by the immune system).

Moreover, it should further be recognized that the microvesicles may be used (even independently of their RNA content) to modify the recipient cell via addition of a lipid component and/or membrane associated polypeptide that is introduced to the recipient cell via fusion with the microvesicle. For example, microparticles may be employed to reduce (via ‘dilution’) the neutral glycosphingolipids, gangliosides, and/or ceramides in platelets, or may be employed to introduce a known membrane associated epitope to which labeled antibodies are already available. Thus, it should be appreciated that recipient cells may be functionally, structurally, and/or compositionally altered, wherein at least some of these effects are achieved without permanent genetic modification.

In vivo uses: In another aspect of the inventive subject matter, RNA in microvesicles may be used as therapeutic agents that at least temporarily alter one or more parameters of a recipient cell. In one preferred example, it is contemplated that microvesicles are derived from nucleated donor cells (and particularly from stem cells), wherein these microvesicles are administered to an injured or otherwise disease tissue (e.g., skin, or bone marrow, or heart, see paper from Nature, Feb. 10, 2005). In such exemplary uses, it is generally preferred that the stem cells are either embryonic stem cells, or omni- or pluripotent stem cells that will develop to a lineage that includes the type of cell that is injured or otherwise diseased. While not wishing to be bound by a particular theory or hypothesis, it is contemplated that the microvesicle will provide RNA and other components (e.g., membrane associated polypeptide, transcription factors, etc.) that will trigger expression of genes associated with cell dedifferentiation and/or cell redifferentiation, or that will prompt the cell to produce factors that attract cells involved in tissue restoration.

Therefore, and among other contemplated uses, microvesicles (especially those from stem cells) may be employed as therapeutic agents, in which the microvesicles are contacted with the an injured or otherwise disease tissue. For example, suitable applications include skin rejuvenation, wound healing, and prevention or reduction of scars at a site of injury or infection. Thus, particularly preferred aspects include those in which microparticles are employed to regenerate tissue that would otherwise scar or necrotize, including hepatic tissue in the treatment of hepatic fibrosis and/or cirrhosis, facial epidermal tissue to treat acne, and cardiac tissue in the treatment of ischemic infarction. It should be noted that microparticles derived from thrombocytes have been suggested as treatment modality for bone injury (see U.S. Pat. App. No. 2004/0082511). However, as thrombocytes are non-nucleated cells, it is questionable that such microvesicles have significant content of RNA. Moreover, due to the relatively high load of thrombogenic factors in platelet derived microparticles, in vivo application for therapeutic use appears problematic as such microparticles dramatically induce and sustain clotting.

In yet further aspects of the inventive subject matter, it is contemplated that the microparticles may also be used as a vaccine in a host, wherein the donor cell is a diseased cell (e.g., neoplastic cell, or HIV infected cell). In such exemplary use, it is expected that the microparticles will contain RNA, membrane associated or cytosolic polypeptides of the diseased cell, which can then be targeted by the immune system. Alternatively, the diseased donor cell can also be subjected to a treatment (ex vivo and/or in vitro using chemicals, biologicals or physical factors such as heat/cold shock, radiation, hypoxia) in which the donor cell becomes changed for example committed to apoptosis or is apoptotic, wherein the microvesicles are then harvested from the so treated donor cell. In such case, it is expected that the microparticles will contain RNA, membrane associated or cytosolic polypeptides of the diseased treated cell, which then can be used for injection to: 1. stimulate immunological response against diseased donor cells (vaccination effect), 2. Induce rejection reaction towards diseased donor cells, 3. Induce resistance to maintain and grow diseased donor cells within the body, and/or can fuse with the diseased cell to induce and/or mark that cell as being committed to apoptosis. Of course, it should be recognized that the donor cells in such contemplated use are preferably from the same organism as the recipient cells. However, where desirable, heterologous donor cells are also deemed suitable (e.g., where the cell is a virally infected disease for vaccination).

Similarly, microparticles are also thought to contribute to immunotolerance where a transplant organ is perfused with microparticles that are isolated from the blood of the organ recipient. Conversely, the organ recipient may also (additionally or alternatively) receive microparticles from the organ donor (typically donor blood, or from donor organ related cells) to induce immunotolerance to the organ in the recipient. For example, microvesicles from the organ or tissue to be transplanted may be injected (s.c., or into the thymus) to the transplant host.

In still further contemplated aspects of the inventive subject matter, microparticles may also be prepared from a particular donor cell known to express a specific cell-surface receptor. It is expected that the microparticle will also comprise that receptor, and that the microparticle preparation may be employed as a soluble receptor to reduce circulating quantities of the corresponding ligand. Use of such microparticles may further be enhanced where it is expected that at least some of the soluble receptor will be deposited into other cells, and where the microparticle further includes an RNA that encodes a polypeptide (or has a regulatory function) associated with the soluble receptor (e.g., G-coupled protein component functionally cooperating with the receptor).

It was further observed that numerous disease states are associated with a substantial increase of microvesicles in the blood stream of a patient. Therefore, it is also contemplated that all compounds and methods that reduce the microvesicles in a (e.g., bloodstream of patients) may also have a desirable effect on a diseased recipient. Among other compounds, preferred compounds to reduce microparticle concentration in vivo include various antibiotics (e.g., Nystatin, Filipin, and similar), statins, flavones, and polyphenols (e.g., apigenin, catechin, resveratrol, curcumin, quercetin) and their analogs, retinoic acids and its analogs, 8-Cl-cAMP, and its analogs, and botanical extracts and compositions and compounds known to affect the cytoskeleton, membrane fluidity and/or membrane ruffling. These compounds could be used together with chemotherapeutics. Regardless of the particular use, it should be noted that the effects precipitated by the microvesicles is under most circumstances a temporary effect, wherein the duration of the effect is predominantly dependent on the half-life time of the RNA in the microvesicles, and/or the membrane turnover rate of the recipient cell.

Microvesicles may also be employed as a component of an device or tissue, wherein the microvesicles may be directly associated with the device or tissue, or indirectly (e.g., via coating layer comprising the microvesicles). For example, culture plates, flasks, catheters, or implanted devices (e.g., stent, cardiac valve prosthesis, etc.) are especially contemplated herein.

### Diagnostic Uses

In a still further preferred use of RNA microparticles, it is contemplated that the RNA of the microparticles may be employed as a diagnostic marker that is reflective of a particular condition of one or more cells. For example, where a neoplastic cell includes a transcript of an oncogene, the oncogene may be detected in the microvesicles shed from the neoplastic cell regardless of the location of the neoplastic cell (e.g., solid or metastatic cell). Microvesicles can be found in all biological materials (e.g., bloodstream, urine, biopsy, etc.), identified, isolated, concentrated and prepared for RNA identification. RNA identification can be done using all using all known manners, including fluorescence labeled antisense oligonucleotides and cytofluorimetric analysis. Alternatively, contemplated RNA need not be limited to an oncogene, but also includes numerous other genes whose sequence and/or quantity can be employed as a disease marker. Suitable RNA markers include those encoding CEA (carcino embryonic antigen) and PSA (prostate specific antigen), v-myc, ki-ras, ha-ras, BRCA-1 and BRCA-2.

It should be particularly recognized that such diagnostic assay provides numerous advantages over commonly practiced assays as the RNA is already present in high copy numbers, in a highly concentrated form (e.g., due to isolation and concentrating microvesicles from biological fluid prior to assaying RNA as compared to serum), and independent from the location of the donor cell. Consequently, especially preferred analytical fluids include biological fluids (e.g., whole blood, urine, saliva) and biopsy samples.

Presence and/or quantity of the RNA in the microvesicles can be determined by numerous manners known in the art, and all known manners are deemed suitable for use herein. However, especially preferred manners include quantitative PCR (qPCR), reverse transcription PCT (rtPCR), and gene-chip-based methods in which the RNA is directly (or indirectly after amplification) hybridized to a solid-phase. Among other things, it is contemplated that such diagnostic methods will assist in the detection and quantification of numerous diseases and conditions that are associated with a detectable RNA, and wherein the RNA in microparticles is associated with a disease (e.g., viral infection, metabolic disease, or neoplastic disease). Among other viral infections, infections with HIV, HCV, influenza or poxviridae are particularly contemplated. Similarly, contemplated diagnostic methods also allow detection of various inherited metabolic diseases (e.g., phenylketonuria (PKU), hypoketotic hypoglycemia, or glycogen storage disease). Contemplated diagnostic methods are also especially useful in the detection of various (optionally residual) neoplastic diseases (e.g., circulating or localized metastases), including prostate cancer, colon cancer, and breast cancer. While preferred qualitative and quantitative diagnostic assays are predominantly based on the RNA content, it should also be appreciated that any diagnostic test may benefit from using isolated or at least partially enriched microvesicle populations to improve sensitivity and/or selectivity.

In yet further aspects of the inventive subject matter, the inventors contemplate that microvesicles may also serve as additional markers for a disease, predisposition, and/or condition, wherein the RNA is not only useful diagnostics but also for characterization or staging of the disease, predisposition, and/or condition. Moreover, the qualitative and/or quantitative relationship between various components of the microvesicles, including lipids, growth factors, ligands, RNA, and other proteins taken as whole may be important as opposed to any one factor. This is especially significant as currently known serum based assays typically cannot distinguish the source of one analyte (and especially as the analyte relates to another analyte). It should further be noted that microvesicles are in many instances associated with a disease and that they are likely to be responsive to various stimuli. Therefore microvesicles should become good candidates as surrogate markers to identify new agents or drugs that stimulate or depress microvesicle production. For example, components of microvesicles other than RNA may also be useful in diagnosis and/or staging of a disease. Among other things, microvesicles generated in vivo from metastatic cancer cells may carry metalloproteinases (at their cell membrane and/or within the microvesicle). Therefore, it is contemplated that microvesicles may be isolated by their cell/organ-specific markers and then be further analyzed for proteolytic activity (e.g., using various fluorescently labeled substrates) as many metastatic cells are know to express metalloproteinases.

Therefore, the inventors contemplate kits, compositions and methods in which a composition includes a microvesicle from a donor cell, wherein the microvesicle encloses at least one RNA. Associated with the composition (e.g., as package insert, or on the container that holds the composition) is an information that provides at least one of a function and an identity of the RNA. The microvesicles may further comprise one or more additional components, including a membrane associated protein, a cytoplasmic protein, a nuclear protein, and a membrane lipid component, and wherein the additional component has a desirable effect (e.g., renders cell identifiable, susceptible to uptake of drug or virus, etc.) on a cell that is contacted with the microvesicle. Most preferably, microparticles and such kits may be employed in methods of modifying a recipient cell in which a plurality of microvesicles is provided that enclose an RNA known to confer a specific effect. The recipient cell is then contacted with the microvesicles in an amount sufficient to deliver the RNA into the recipient cell to thereby modify the recipient cell. Viewed from another perspective, suitable kits may also include those comprising a microvesicle from a nucleated donor cell, and further including an information associated with the composition to combine the microvesicle with a recipient cell in vitro to achieve a desired effect in the recipient cell. Such and other kits may therefore include an information associated with the composition to use the composition for an in vitro use (the term “in vitro” as used herein also includes ex vivo).

Consequently, contemplated composition and kits may be marketed by providing a composition comprising a microvesicle from a donor cell, wherein the microvesicle has at least one RNA, and by providing information associated with the composition that provides at least one of a function and an identity of the RNA. Where microvesicles are employed to in vitro, it is also contemplated that the microparticles may be provided in a composition of a kit along with an information associated with the composition to combine the microvesicle with an in vitro growth medium to achieve a desired effect. Thus, especially preferred growth media include powdered media formulations that comprise a preserved microvesicle.

In still further contemplated aspects, a method of diagnosing a condition in a mammal has one step in which a sample is provided that comprises a plurality of microvesicles isolated from the mammal, wherein at least one of the microvesicles encloses an RNA that is a known etiologic component of the condition. In another step, the presence and/or quantity of the RNA in the sample is determined, and the presence and/or quantity of the RNA is optionally associated with a condition in the mammal. The term “known etiologic component of the condition” refers to a RNA component that has regulatory and/or protein-encoding function in a cell, wherein the regulatory and/or protein-encoding function is contributes to the condition. For example, contemplated RNA components include RNA corresponding to an oncogene, RNA encoding a mutated gene that leads to a dysfunctional polypeptide that is involved in a condition (e.g., mutation/deletion in dystrophin gene), and/or RNA corresponding to a viral nucleic acid (e.g., HIV, HCV, or HPV).

Based on several publications and patents, it is suggested that cells change with age with respect to physiology. Here the inventors contemplate that microvesicle formation and content will change accordingly, and that such change may be used to monitor the aging processes. Alternatively, the microvesicle production and/or RNA content may be employed as a surrogate marker to identify drugs, foods, and/or treatments that reduce generation of microvesicles within the body, which in turn may be advantageous to a persons health. Alternatively, drugs may be selected and/or administered to inhibit microvesicle generation and/or fusion similar to VEIs (virus entry inhibitors), or dialysis may be employed to reduce the microvesicle concentration in a patient. Thus, and viewed from yet another perspective, it is contemplated that the generation and/or content of microvesicles may be time and stimulant dependent, and therefore be useful in pharmacology and toxicology studies (e.g., acting as s surrogate marker).

As microvesicles are likely to represent a heterogeneous population when isolated from an organism, it should be appreciated that isolated microvesicles can be further analyzed by virtue of one or more markers (e.g., via FACS, or affinity separation). Such differentially isolated microvesicles may then be employed as therapeutic or diagnostic agents with improved specificity. Of course, the difference in populations may be reflective of a disease or disease state, the specific donor cell, and/or the age of the donor cell. For example, subpopulation of cancer cells from one cancer lesion/tumor have been reported to be in large part characteristic to the tumor's tumorigenicity, aggressiveness, metastatic potential etc. Such cells can now be identified by cell surface markers and tested in vivo or in vitro for differentiating characteristics (clonogenic growth, chemotaxis assays, in vivo tumorigencity assays, invastion assays etc). It is important to recognize that microvesicles from tumor cells derived from a single tumor may indeed be different as they arise from different subsets of cells from that tumor and that the character and analysis of these microvesicles may allow for better investigation, diagnostics, and drug targets.

### EXPERIMENTS

### Isolation and RNA determination from Embryonic Stem Cell Microvesicles

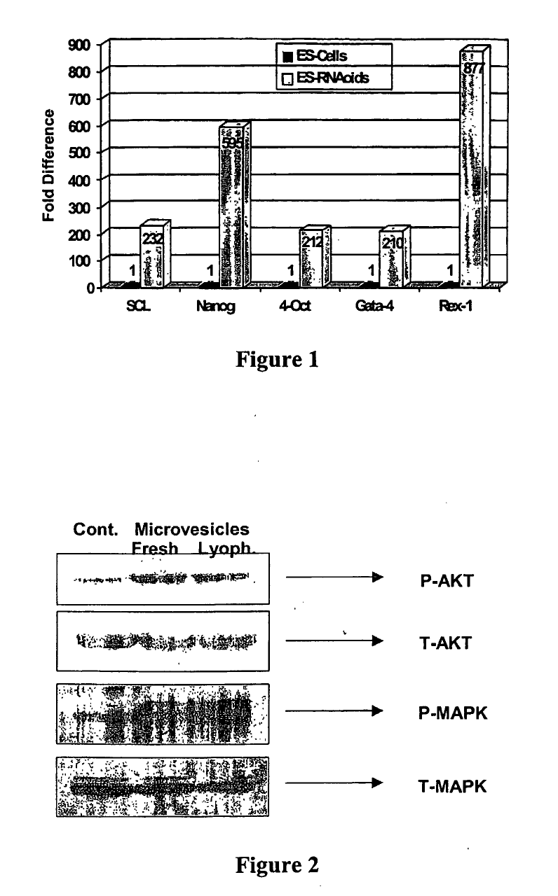

Microvesicles were isolated from embryonic cells (embryonic cell line) and from human platelets by high speed centrifugation using standard protocols. For isolation of RNA from the microvesicles, equal amount of microvesicles as compared to stem cells were employed. Primers for selected genes were chosen to amplify RNA for various transcription factors, including SCL, Nanog, 4-OCT, GATA-4, Rex-1. FIG. 1 depicts the results of the real-time PCR in which selected RNAs from stem cells as well as from microvesicles were amplified. Microvesicles were typically used in the experiments below at a concentration of about 10²-10⁶ per cell in culture.

As can be clearly seen, it is shown, amplification of cDNA corresponding to selected transcription factors is substantially higher in microvesicles than in parental embryonic cells. This provides convincing data to support the discovery that microvesicles not only include RNA, but that the RNA concentration in microvesicles is substantially higher than in the parental donor cells.

### RNA Stability in Fresh and Preserved Microvesicles

Microvesicles were prepared fresh from stem cells as above and one portion of the microvesicles was freeze-dried, while the other portion was maintained at 4° C. After one day, RNA was extracted from the microvesicles using standard RNA isolation kits (e.g., Quiagen) and selected target RNAs were amplified and subjected to gel electrophoresis. FIG. 2 depicts an autoradiograph indicating stability of the RNA in the lyophilized microvesicle preparation. Using comparable assays, RNA content (qualitatively and quantitatively) of any microvesicle can be easily monitored and asserted.

### RNA Transfer by Microvesicles

Since the development of embryoid bodies is, among other things, regulated by close cell-to-cell interactions between embryonic stem cells (ES) by membrane-expressed and other molecules, it is contemplated that ES-derived MV (ESMV) could have or could lead to expression of one or more stem-cell specific molecules that affect the biology of target cells and play an important role in cell-cell communication. To test this hypothesis ESMV were isolated from conditioned media harvested from murine ES cells (ES-D3) and human ES cells (CCTL14), washed out the soluble factors by centrifugation and analyzed expression of mRNA for early transcription regulatory proteins in both ESMV and expanded cells (Oct-4, Nanog, Rex-1, SCL).

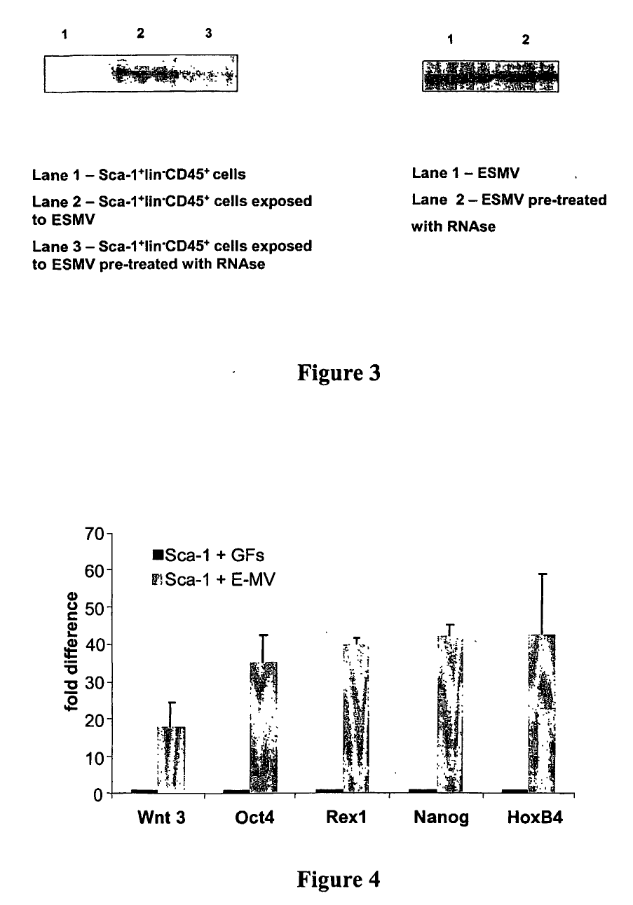

Western blot and qPCR analysis was done for cells subjected to mRNA (here: Oct-4) transfer via ESMV. Here, the recipient cells were bone marrow (BM)-derived Sca-1+lin-CD45+ cells. The results clearly demonstrate that the microvesicles enclose mRNA as the Oct-4 RNA remains intact upon Rnase treatment of the microvesicles as depicted in the right panel of FIG. 3. The left panel clearly shows that the untreated recipient cells are devoid of Oct-4 expression, but do express Oct-4 upon incubation of the cells with ESMV known to include mRNA encoding Oct-4.

### Use of ESMV to Stimulate and Expand Hematopoietic Stem Cells

Remarkably, the mRNA transferred to the recipient cell via MV provided (directly or indirectly) substantial effect on other markers as well and FIG. 4 depicts an exemplary panel of growth factor expression in recipient cells expanded with ESMV. Here, expression of mRNA for Wnt-3 and selected early transcription factors (Oct4, Rex 1, Nanog, HoxB4) were measured during expansion (5th day (n=4)) of murine bone marrow-derived Sca-1+kit+lin-hematopoietic stem cells. The bars in FIG. 4 depict fold difference in expression of selected growth factors in the recipient cells, wherein the control is cells grown in medium using growth factors (+GFs), and wherein E-MV denotes treatment with microvesicles from murine embryonic stem cells.

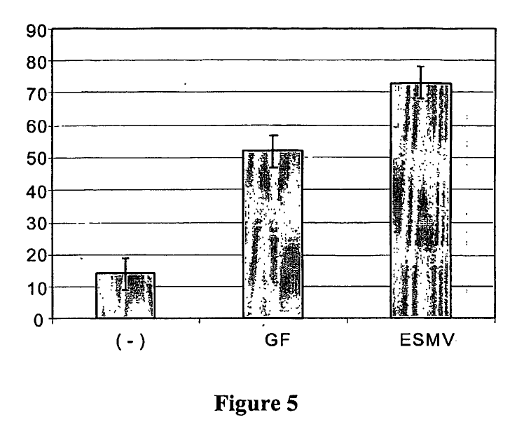

Remarkably, expansion of the stem cells was substantially affected as well as depicted in FIG. 5. Here, untreated cells were used as control, while cells treated with growth factors were labeled GF and cells treated with the microvesicles (see above) were denoted ESMV. Clearly, at day 12, the number of colonies from the ESMV treated cells substantially increased whereas the number of colonies from the control and growth factor treatment remained at an expected level. Surprisingly, cell proliferation was lower in the ESMV treated group as compared to the growth factor (GF) treated group. In yet a further surprising finding (data not shown), the inventors discovered that the microvesicles are also effective to improve cell viability in cryopreservation and recovery from cryopreservation.

Thus, specific embodiments and applications of RNA-containing microvesicles have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

1-32. (canceled)

33. A method of diagnosing a disease or predisposition in a mammal, comprising:
providing a sample comprising a plurality of microvesicles obtained from the mammal, wherein at least one of the microvesicles encloses an RNA that is a known marker of the disease or predisposition;
differentially isolating a sub-population of the microvesicles using a cell and/or organ-specific marker;
determining at least one of a presence and a quantity of the RNA in the sample; and
correlating the at least one of the presence and the quantity of the RNA with the disease or predisposition.

34. The method of claim 33 wherein the sample is isolated from a biological fluid of the mammal.

35. The method of claim 33 wherein the step of determining of the at least one of the presence and the quantity of the RNA comprises at least one of an RT-PCR, a Q-PCR, and a hybridization of the RNA on a solid phase.

36. The method of claim 33 wherein the disease or predisposition is a metabolic disease, an intoxication, or a neoplastic disease.

37. The method of claim 36 wherein the metabolic disease is selected from the group consisting of phenylketonuria, hypoketotic hypoglycemia, and glycogen storage disease, and wherein the neoplastic disease is selected from the group consisting of prostate cancer, colon cancer, and breast cancer.

38. The method of claim 33 wherein the step of differentially isolating employs at least one of FACS and affinity separation.

39. The method of claim 33 further comprising a step of using the microvesicles as surrogate marker to identify a drug that stimulates or depresses microvesicle production.

40. The method of claim 33 further comprising a step of analyzing qualitative and/or quantitative relationship between the RNA and a protein in the microvesicle.
